(19) United States
(12) Patent Application Publication
(10) Pub. No.: US 2009/0043158 A1
(43) Pub. Date: Feb. 12, 2009

(54) Conduit introducer, conduit assembly and conduit-implanting device

(76) Inventor: Wei-Chen Hon, Tainan City (TW)

Correspondence Address: BUCKNAM AND ARCHER, 1077 NORTHERN BOULEVARD, ROSLYN, NY, 11576, US

(21) Appl. No.: 12/006,405

(22) Filed: Jan. 2, 2008

(30) Foreign Application Priority Data
Aug. 10, 2007 (TW) 096129488

(51) Int. Cl. A61B 1/00

(52) U.S. Cl. 600/114

(57) ABSTRACT

A conduit-implanting device includes a conduit introducer and a conduit assembly. The conduit introducer includes: a rod body having an inserting end located at a lower side of the rod body; an inflatable section disposed in the proximity of the inserting end; and an injection port for injecting liquid into the inflatable section. The conduit assembly includes a hollow tube body, a plunger and a plunger rod. The plunger includes an upper end face connected with the plunger rod, and a lower end face being an abutting end. The plunger moves upward in the hollow tube body by operating the plunger rod.

### BACKGROUND OF THE INVENTION

1. Field of the Invention

This invention relates to a conduit-implanting device, and more particularly to an implanting device of working conduit used for endoscopic surgery.

2. Brief Description of the Prior Art

A Taiwanese Patent No. M283609 discloses a working conduit for intracranial endoscopy, which include an outer conduit and an inner conduit. In order to remove intracranial hemorrhage, there is a need to temporarily implant or insert the working conduit for endoscopic operation in brain organization. Firstly, the inner conduit is placed within the outer conduit, and then the inner conduit and the outer conduit are inserted together in the brain organization. Next, the inner conduit is removed so that the outer conduit is used only as the working conduit for endoscope.

Apparently, the outer conduit has sufficient inner diameter so as to receive the endoscope and/or other tools needed for operation, such as a suction tube. This will make the outer diameter of the outer conduit bigger than the diameter of the endoscope. When the inner conduit and the outer conduit are inserted together in the brain organization, the squeezed area of the brain organization is equal to the area surrounded by the outer peripheral surface of the outer conduit.

However, the brain organization is very vulnerable and not easy to recover once gets hurt. If the squeezed area is too large, it is possible to harm the brain organization. Therefore, a tool capable of reducing the squeezed area of the brain organization during conduit implantation, which is able to make the squeezed area of the brain organization even smaller than the cross section of the endoscope, is anticipated.

### SUMMARY OF THE INVENTION

In view of the above concern, this invention proposes a conduit introducer, a conduit assembly and a conduit-implanting device, in which the conduit-implanting device includes the conduit introducer and the conduit assembly. The conduit introducer includes: a rod body which has an inserting end located at an lower side of the rod body; an inflatable section disposed in the proximity of the inserting end; and an injection port for injecting liquid into the inflatable section. The conduit assembly includes a hollow tube body, a plunger and a plunger rod. The plunger includes an upper end face connected with the plunger rod, and a lower end face, which is an abutting end. The plunger moves upwards in the hollow tube body by operating the plunger rod.

Preferably, the conduit introducer further includes a valve body provided between the inflatable section and the injection port.

Preferably, the inflatable section of the conduit introducer is preferably formed by a resilient membrane.

Preferably, the hollow tube body of the conduit assembly is made of transparent material for easy observation.

Preferably, the plunger of the conduit preferably has a plurality of air-permeable holes for communicating with the upper end face and the lower end face of the plunger.

Preferably, the plunger of the conduit is composed of an inflatable air bag.

Preferably, the inserting end of the conduit introducer and/or the abutting end of the plunger are in form of semi-spherical shape, arc-like shape, circular arc shape or conic shape with rounded tip so as to reduce friction during insertion.

Preferably, wing sections can be provided near the upper ends of the rod body of the conduit introducer and/or of the hollow tube body of the conduit assembly for hand gripping.

When the conduit is implanted to conduct endoscopic operation, the conduit introducer is firstly inserted into the organization. Fluid (for example, air or water) is then injected into the inflatable section through the injection port to allow inflation of the inflatable section, so that the brain organization can be braced up. Then, the conduit introducer may be removed directly or removed after discharge of the fluid in the inflatable section. Before the braced opening on the organization is closed, the conduit assembly is inserted, in a manner that the plunger is positioned at the front end of the hollow tube body, along the braced opening. Finally, the plunger is removed from the hollow tube body and thus the hollow tube body is remained only to serve as the working conduit for endoscope.

Compared with that of the prior art, the squeezed area of organization can be reduced to a minimum by implanting the working conduit of endoscope in accordance with the present invention.

### BRIEF DESCRIPTION OF THE ACCOMPANYING DRAWINGS

FIG. 1 is a view showing an embodiment of the conduit-implanting device of the present invention;

FIG. 2a to 2e are the schematic views showing the operation processes of implanting the working conduit of endoscope by the conduit-implanting device of the present invention; and

FIG. 3 is a schematic view showing the endoscopic surgery by means of the working conduit implanted by the conduit-implanting device of the present invention.

### DETAILED DESCRIPTION OF THE PREFERRED EMBODIMENTS

The technical features of the present invention will become more apparent by the detailed description of the following preferred embodiments of the present invention in conjunction with the accompanying drawings. The embodiments are preferred embodiments purely for the convenience of description only, and are not intended to be restrictive of the scope of the present invention. It is noted that the drawings are for the convenience of description only and hence are not plotted in full scale.

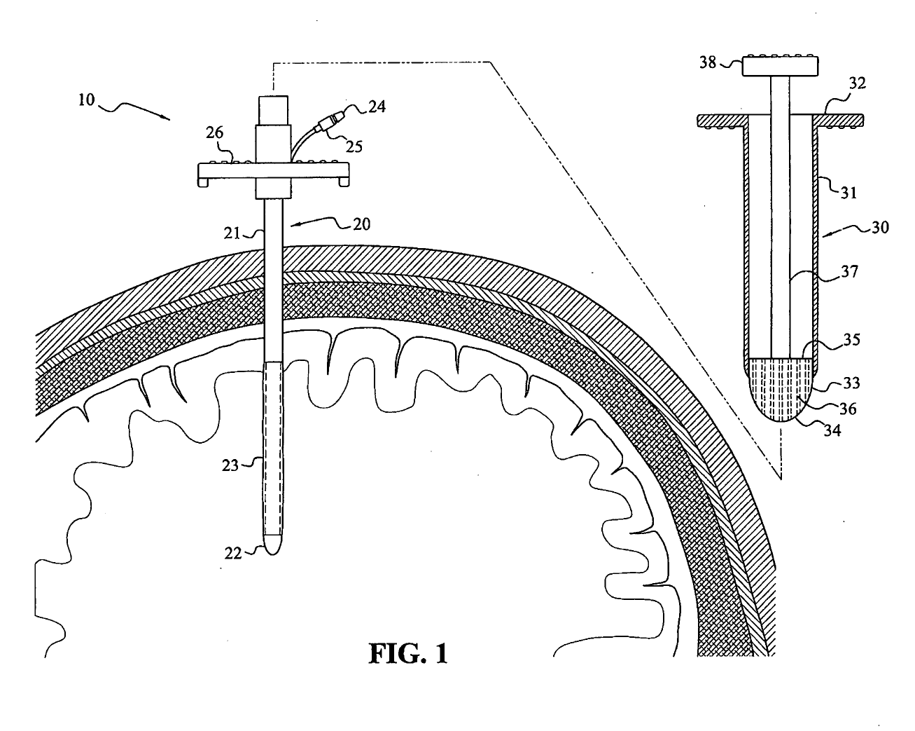

Firstly, please to FIG. 1. An embodiment of the conduit-implanting device of the present invention is shown. The whole conduit-implanting device is designated by the reference numeral 10. The conduit-implanting device 10 includes a conduit introducer 20 and a conduit assembly 30.

The conduit introducer 20 includes a rod body 21, which has an inserting end 22 located at a lower side thereof. An inflatable section 23 having a predetermined length is provided to closely abut with the inserting end 22. The inflatable section 23 is formed by a resilient membrane. For example, the resilient membrane is wrapped around the outer periphery of the rod body so as to form an inflatable air bag. Furthermore, an injection port 24 is provided on an upper side of the rod body 21 to communicate with the inflatable section 23 such that fluid (for example, air or water) is injected into the inflatable section 23 through the injection port 24. The inflatable section 23 can be inflated by the injected fluid. The conduit introducer 20 includes a wing section 26 located near an upper end of the rod body 21 and facilitating hand to grip or operate.

Additionally, a valve body 25 is provided between the injection port 24 and the inflatable section 23 for controlling the inflation and deflation of the inflatable section 23. Alternatively, an injection port 24 having a normally closed opening is formed by using a resilient tube with thicker tube wall so as to possess the function of valve.

The conduit assembly 30 includes a hollow tube body 31, a plunger 33 and a plunger rod 37. The plunger 33 includes an upper end face 35 connected with the plunger rod 37, and a lower end face 34, which is an abutting end. The plunger 33 moves upwards in the hollow tube body 31 by operating the plunger rod 37 and is removed from the hollow tube body 31. The plunger 33 has a plurality of air-permeable holes 36 which communicate with the upper ends face 35 and the lower end face 34 of the plunger 33 so as to avoid negative or vacuum pressure formed on the lower end face 34 during the removal of the plunger 33. Wing sections 32 and 38 are respectively provided at an upper end portion of the hollow tube body 31 and the plunger rod 37 to facilitate hand to grip and operate.

Another method (not shown) for preventing the formation of negative or vacuum pressure on the lower end face 34 during the removal of the plunger 33 is to use an inflatable air bag as the plunger 33. The inflatable air bag in a fluid-charged state could function as an ordinary plunger. When the plunger formed by the inflatable air bag is removed, the inflatable air bag is deflated first and then the plunger is removed. In such manner, negative or vacuum pressure formed on the lower end face during the removal of the plunger can also be avoided.

Preferably, the inserting end 22 of the conduit introducer 20 and the abutting end of the plunger 33 are formed to have a smooth shape without cute corner, such as semi-spherical shape, arc-like shape or conic shape with rounded tip so as to reduce friction during the insertion. The lower end of the hollow tube body 31 can also be chamfered so as to avoid scraping organization.

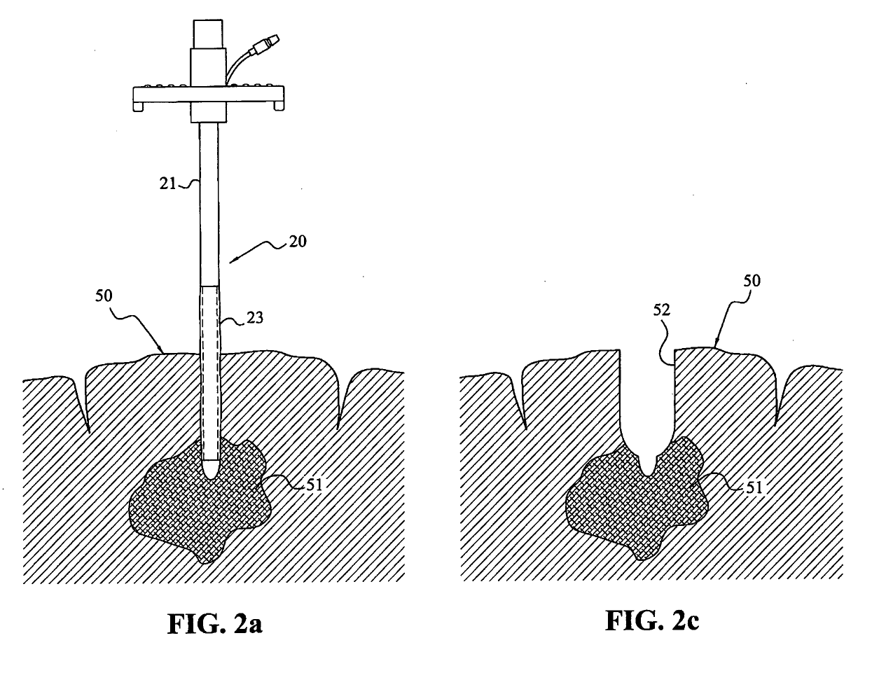

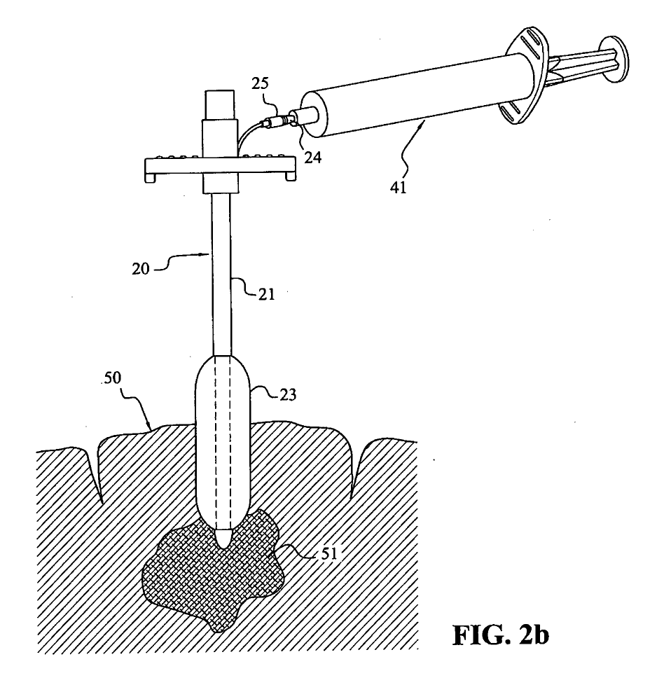

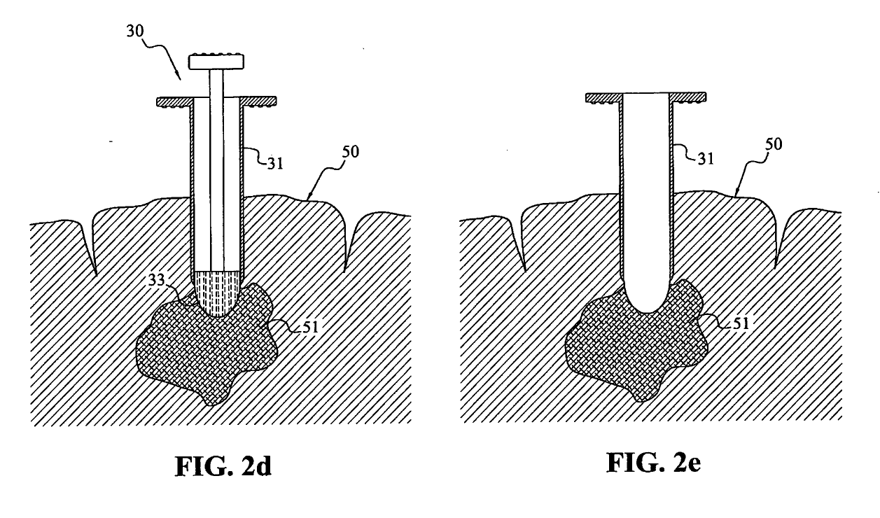

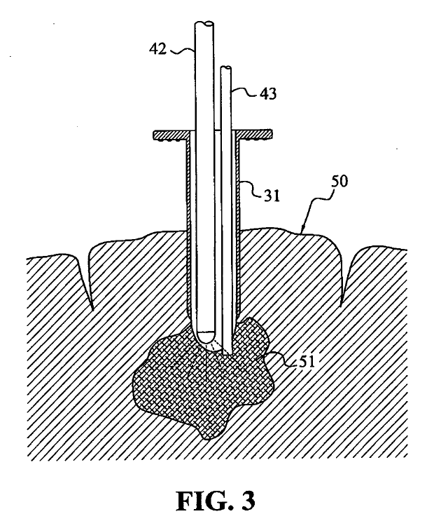

Next, the procedures of using the conduit-implanting device of the present invention are described in an example of endoscopic surgery for clearing the intracelebral ecchymosis. FIG. 2a to 2c show the operation processes of temporarily implanting the working conduit into brain organization in an endoscopic surgery by the conduit-implanting device of the present invention.

In order to remove the intracranial hemorrhage 51 in the brain organization 50, the long and narrow conduit introducer 20 is inserted into the brain organization under the state that the brain organization is exposed due to surgery operation, as shown in FIG. 2a. As shown in FIG. 2b, air is injected from the injection port 24 into the inflatable section 23 by a syringe 41 such that the brain organization 50 is braced up due to the expansion of the inflatable section 23. As shown in FIG. 2c, the conduit introducer 20 is removed after the inflatable section 23 is deflated so as to form an opening 52 atop the brain organization 50. Before the braced opening 52 is closed, the conduit assembly 30 is inserted in the brain organization 50 along the braced opening 52 in the manner that the plunger 33 is disposed at the front end of the hollow tube body 31. As shown in FIG. 2d, the plunger 33 is removed from the hollow tube body 31 and the hollow tube body 31 is finally remained to serve as the working conduit for endoscopic surgery. As shown in FIG. 2e, the working conduit for endoscopic surgery operation temporarily implanted with the above method by the conduit-implanting device according to the present invention is capable of remarkably reducing squeezed area of the brain organization.

FIG. 3 is a schematic view showing the endoscopic surgery performed by using the hollow tube body 31 as the working conduit. As shown in FIG. 3, the endoscope 42 and a suction tube 43 pass through the hollow tube body 31 so as to remove the intracranial hemorrhage 51 in the brain organization 50.

Another advantage of using the conduit implanting device in accordance with the present invention lies in that as a plurality of air-permeable holes are provided on the plunger, negative or vacuum pressure will not happen between the plunger and the brain organization when the plunger is pulled out. Therefore, the brain organization won't be drawn out while the plunger is pulled out.

Additional advantages and modifications will readily occur to those skilled in the art. Therefore, this invention in its broader aspects is not limited to the specific details and representative embodiment shown and described herein. Accordingly, various modifications and variations may be made without departing from the spirit and scope of the general inventive concept as defined by the appended claims and their equivalents.

1. A conduit-implanting device for endoscope, comprising:
a conduit introducer having a rod body which has an inserting end located at the lower side of the rod body; an inflatable section disposed in the proximity of the inserting end; and an injection port for injecting liquid into the inflatable section; and a conduit assembly having a hollow tube body, a plunger and a plunger rod, wherein said plunger comprises an upper end face connected with said plunger rod and a lower end face being an abutting end, and said plunger moves upwards in said hollow tube body by operating said plunger rod.

2. A conduit-implanting device for endoscope as claimed in claim 1, wherein said conduit introducer further comprises a valve body provided between said inflatable section and said injection port.

3. A conduit-implanting device for endoscope as claimed in claim 2, wherein said injection port and said valve body are provided in the proximity of an upper end of said rod body.

4. A conduit-implanting device for endoscope as claimed in claim 1, wherein a wing section is provided near an upper end of said rod body of said conduit introducer.

5. A conduit-implanting device for endoscope as claimed in claim 1, wherein said inflatable section is formed by a resilient membrane.

6. A conduit-implanting device for endoscope as claimed in claim 1, wherein said hollow tube body is made of transparent material.

7. A conduit-implanting device for endoscope as claimed in claim 1, wherein said plunger has a plurality of air-permeable holes for communicating with said upper end face and said lower end face of said plunger.

8. A conduit-implanting device for endoscope as claimed in claim 5, wherein a wing section is provided at an upper end of said hollow tube body and said rod body respectively.

9. A conduit-implanting device for endoscope as claimed in claim 1, wherein said inserting end of said conduit introducer and said abutting end of said plunger are in form of semi-spherical shape, arc-like shape, circular arc shape or conic shape with rounded tip.

10. A conduit-implanting device for endoscope as claimed in claim 1, wherein said plunger is constituted by inflatable air bag.

11. A conduit introducer, comprising:
a rod body, having an inserting end located at a lower side of said rod body;
an inflatable section disposed in the proximity of said inserting end and formed by a resilient membrane; and
an injection port for injecting liquid into said inflatable section.

12. A conduit introducer as claimed in claim 11, wherein said inserting end is in form of a circular arc shape or a conic shape with rounded tip.

13. A conduit introducer as claimed in claim 11, wherein said conduit introducer includes a valve body provided between said inflatable section and said injection port.

14. A conduit introducer as claimed in claim 11, wherein a wing section is provided near an upper end of the rod body.

15. A conduit assembly, comprising: a hollow tube body, a plunger and a plunger rod, wherein said plunger comprises an upper end face connected with said plunger rod and a lower end face being an abutting end; said plunger moves upwards in said hollow tube body by operating said plunger rod, and said plunger has a plurality of air-permeable holes for communicating with said upper end face and said lower end face of said plunger.

16. A conduit assembly as claimed in claim 15, wherein said hollow tube body is made of transparent material.

17. A conduit assembly as claimed in claim 15, wherein said abutting end of said plunger is in form of semi-spherical shape, arc-like shape, circular arc shape or conic shape with rounded tip.

18. A conduit assembly as claimed in claim 15, wherein a wing section is provided at an upper end of said hollow tube body and said rod body respectively.

19. A conduit assembly as claimed in claim 15, wherein said plunger is constituted by inflatable air bag.
